(19) United States
(12) Patent Application Publication
(10) Pub. No.: US 2025/0009644 A1
(43) Pub. Date: Jan. 9, 2025

(54) LONG-ACTING INJECTABLE IN SITU FORMING BIODEGRADABLE IMPLANTS COMPRISING NON-STEROIDAL ANTI-INFLAMMATORY DRUGS

(71) Applicant: University of Connecticut, Farmington, CT (US)

(72) Inventors: Diane Burgess, Farmington, CT (US); Xiaoyi Wang, Farmington, CT (US)

(73) Assignee: University of Connecticut, Farmington, CT (US)

(21) Appl. No.: 18/708,433

(22) PCT Filed: Nov. 30, 2022
(86) PCT No.: PCT/US2022/051358

§ 371 Date: May 8, 2024

Related U.S. Application Data
(60) Provisional application No. 63/285,169, filed on Dec. 2, 2021.

(51) Int. Cl. A61K 9/00; A61K 31/192; A61K 31/5415; A61K 47/34

(52) U.S. Cl. CPC A61K 9/0002; A61K 9/0024; A61K 31/192; A61K 31/5415; A61K 47/34

(57) ABSTRACT

The present disclosure provides compositions, systems, pre-filled delivery devices, and methods for developing injectable compositions useful for in situ forming solid depots for the sustained release of non-steroidal anti-inflammatory drug NSAIDs over a period of days to several months for use in treating or preventing inflammation. The compositions comprise a biodegradable polymer such as poly(lactic-co-glycolic acid) (PLGA), a solvent such as N-Methyl-2-pyrrolidone, and a NSAID such as naproxen, meloxicam. The compositions may optionally comprise an additive such as hydrogenated soy L-α-phosphatidylcholine (HSPC) or a second active agent. The depot material will be robust to exist within the desired site for a period similar to or greater than the planned period of drug delivery.

### RELATED APPLICATION INFORMATION

This application claims priority to U.S. Provisional Application No. 63/285,169, filed on Dec. 2, 2021, the contents of which are herein incorporated by reference.

### FIELD

The present disclosure relates to compositions, systems, pre-filled delivery devices, and methods for in situ forming biodegradable drug depots comprising non-steroidal anti-inflammatory drugs.

### BACKGROUND

Non-steroidal anti-inflammatory drugs (NSAIDs) are widely used to treat inflammation and are required for long-term use in many patients, for example for symptoms associated with osteoarthritis and other chronic musculoskeletal conditions. Long-term use, however, can be problematic, as the most commonly used dosage forms, tablets and suspensions, have been found to cause gastrointestinal bleeding, particularly for patients with a history of stomach ulcers or bleeding. Although NSAID injections and suppository dosage forms are currently available, they are associated with poor compliance and can only be used for short term pain relief.

### SUMMARY

Disclosed herein are injectable depot compositions or systems comprising a non-steroidal anti-inflammatory drug (NSAID), a biodegradable polymer, and a solvent. In some embodiments, the compositions and systems comprise 30-40% by weight of the biodegradable polymer. In some embodiments, the compositions and systems comprise 10-20% by weight of the NSAID.

In some embodiments, the biodegradable polymer comprises an aliphatic polyester. In some embodiments, the biodegradable polymer comprises poly(lactic-co-glycolic acid) (PLGA). In some embodiments, the PLGA comprises at least 50% (e.g., at least 55%, at least 60%, at least 65%, at least 70%, at least 75%, at least 80%, at least 85%, at least 90%, or at least 95%) lactic acid units.

In some embodiments, the solvent comprises N-methyl-2-pyrrolidone.

In some embodiments, the NSAID comprises naproxen. In some embodiments, the compositions or systems comprise naproxen, PLGA, and N-methyl pyrrolidone.

In some embodiments, the NSAID comprises meloxicam. In some embodiments, the compositions or systems comprise meloxicam, PLGA, and N-methyl pyrrolidone.

In some embodiments, the compositions or systems further comprise an additive, a preservative, a stabilizer, a buffer, an antioxidant, or a combination thereof. In some embodiments, the compositions or systems further comprise an additional pharmaceutically active agent.

Further disclosed herein are pre-filled delivery devices comprising the compositions or systems disclosed herein.

Also disclosed herein are methods of treating a disease or disorder comprising administering an effective amount of a composition or system disclosed herein to a subject in need thereof.

Other aspects and embodiments of the disclosure will be apparent in light of the following detailed description and accompanying figures.

### BRIEF DESCRIPTION OF THE DRAWINGS

FIG. 1A is an image of prepared naproxen in situ forming implants.

FIG. 1B is a graph of in vitro release profiles of naproxen from in situ forming implants prepared using different PLGAs (n=3). USP apparatus 2 (paddle) method was used in this in vitro release test (37° C., 50 rpm, release media: 100 mL of pH 7.4 Phosphate Buffer Saline containing sodium azide). FN1-6, n=3.

FIG. 2A is an image of prepared meloxicam in situ forming implants.

FIG. 2B is a graph of in vitro release profiles of meloxicam from in situ forming implants prepared using different PLGAs (n=3). USP apparatus 2 (paddle) method was used in this in vitro release test (37° C., 50 rpm, release media: 300 mL of pH 7.4 Phosphate Buffer Saline containing sodium azide). FM1, n=1; FM2, n=3.

### DETAILED DESCRIPTION

Disclosed herein are injectable compositions useful for in situ forming solid depots for the sustained release of NSAIDs over a period of days to several months for use in treating or preventing inflammation. The compositions comprise a biodegradable polymer such as poly(lactic-co-glycolic acid) (PLGA), a solvent such as N-Methyl-2-pyrrolidone, and a NSAID such as naproxen, meloxicam. The compositions may optionally comprise an additive such as hydrogenated soy L-α-phosphatidylcholine (HSPC) or a second active agent. The compositions may be formed immediately prior to administration, or alternatively, may exist and be stored as solutions or suspensions.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the terms “providing,” “administering,” and “introducing” are used interchangeably herein and refer to the placement of the compounds and/or compositions of the present disclosure into a subject by a method or route which results in at least partial localization of the compound and/or composition to a desired site. The compound and/or compositions can be administered by any appropriate route which results in delivery to a desired location in the subject.

The term “prolonged release” herein refers to any formulation or dosage form that comprises an active drug and which is formulated to release the drug for longer duration of time and to provide a longer duration of pharmacological response after administration of the dosage form than is ordinarily not experienced after administration of a corresponding immediate release formulation comprising the same drug in the same amount. Prolonged release formulations include, inter alia, those formulations described elsewhere as “controlled release,” “delayed release,” “sustained release,” “extended release,” “programmed release,” “time release,” and/or “rate controlled” formulations or dosage forms.

A “subject” or “patient” may be human or non-human and may include, for example, animal strains or species used as “model systems” for research purposes. Likewise, subject may include either adults or juveniles (e.g., children), males or females. In some embodiments, the subject is an adult. In some embodiments, the subject is a female. Moreover, subject may mean any living organism, preferably a mammal (e.g., human or non-human) that may benefit from the administration of compounds and/or compositions contemplated herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one aspect of the methods provided herein, the mammal is a human. In some embodiments, the subject is an adult human.

As used herein, “treat,” “treating,” and the like means a slowing, stopping, or reversing of progression of a disease or disorder. The term also means a reversing of the progression of such a disease or disorder. As such, “treating” means an application or administration of the methods, compounds, or compositions described herein to a subject, where the subject has a disease or a symptom of a disease, where the purpose is to cure, heal, alleviate, relieve, alter, remedy, ameliorate, improve, or affect the disease or symptoms of the disease.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. INJECTABLE DEPOT COMPOSITION OR SYSTEM

The present disclosure provides compositions, systems, and delivery devices for an injectable drug depot. The injectable depot forms a solid depot in situ thereby facilitating implantation and retention in a desired site. The depot material will be robust to exist within the desired site for a period of time similar to or greater than the planned period of drug delivery.

The compositions and systems comprise a non-steroidal anti-inflammatory drug (NSAID), a biodegradable polymer, and a solvent.

The NSAID may be any non-steroidal anti-inflammatory compound. NSAIDs are categorized by virtue of their ability to inhibit cyclooxygenase. NSAIDs are generally classified based on their chemical structure or mechanism of action. Cyclooxygenase 1 (COX-1) and cyclooxygenase 2 (COX-2) are two major isoforms of cyclooxygenase and most standard NSAIDs are mixed inhibitors of the two isoforms, but selective COX-1 or COX-2 inhibitors are also known.

Most standard NSAIDs fall within one of the following five structural categories: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxicam, sudoxicam, and isoxicam.

Certain exemplary COX-2 inhibitors include celecoxib (SC-58635), rofecoxib, DUP-697, flosulide (CGP-28238), meloxicam, 6-methoxy-2 naphthylacetic acid (6-MNA), MK-966, nabumetone (prodrug for 6-MNA), nimesulide, NS-398, SC-5766, SC-58215, and T-614.

In some embodiments, the NSAID is selected from the group consisting of aceclofenac, acemetacin, actarit, alclofenac, alminoprofen, amfenac, aloxiprin, aminophenazone, antraphenine, aspirin, azapropazone, benorilate, benoxaprofen, benzydamine, butibufen, celecoxib, chlorphenoxamine, choline salicylate, clometacin, dexketoprofen, diclofenac, diflunisal, emorfazone, epirizole; etodolac, etoricoxib, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidine, loxoprofen, lumiracoxib, mefenamic acid, meloxicam, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protizinic acid, rofecoxib, salicylamide, salsalate, sulindac, suprofen, tiaramide, tinoridine, tolfenamic acid, valdecoxib, zomepirac, and combinations thereof.

The composition or system may comprise 10-90% by weight of the NSAID. In some embodiments, the composition or system comprises 10-20% by weight, 10-30% by weight, 10-40% by weight, 10-50% by weight, 10-60% by weight, 10-70% by weight, 10-80% by weight, 20-30% by weight, 20-40% by weight, 20-50% by weight, 20-60% by weight, 20-70% by weight, 20-80% by weight, 20-90% by weight, 30-40% by weight, 30-50% by weight, 30-60% by weight, 30-70% by weight, 30-80% by weight, 30-90% by weight, 40-50% by weight, 40-60% by weight, 40-70% by weight, 40-80% by weight, 40-90% by weight, 50-60% by weight, 50-70% by weight, 50-80% by weight, 50-90% by weight, 60-70% by weight, 60-80% by weight, 60-90% by weight, 70-80% by weight, 70-90% by weight, or 80-90% by weight, of the NSAID.

Biodegradable materials are natural or synthetic in origin and are degraded in vivo, either enzymatically or non-enzymatically or both, to produce biocompatible, toxicologically safe by-products which are further eliminated by the normal metabolic pathways. The biodegradable polymer may comprise a synthetic biodegradable polymer, including, for example, relatively hydrophobic materials such poly(lactic-co-glycolic acid) (PLGA), polyanhydrides, and others, and naturally occurring polymers, for example complex sugars (e.g., hyaluronan, chitosan) and inorganics (hydroxyapatite). Preferably, the biodegradable polymer degrades in vivo over a period of less than a year.

The compositions or systems may comprise a biodegradable polymer or polymers that facilitate immediate or prolonged release of the NSAID. Thus, in some embodiments, the compositions or systems described herein facilitate prolonged release of the NSAID. The prolonged release of the NSAID may or may not be linear. For example, the prolonged release may comprise a bolus release on day 1.

Some of the disclosed systems and compositions will release between 10 and 50% of the NSAID on day 1 and all or substantially all of the NSAID by day 10. Some of the disclosed systems and compositions will release between 10 and 20% of the NSAID on day 1, between 50 and 80% of the NSAID by day 10, and all or substantially all of the product by day 30. Some of the disclosed systems and compositions will release between 10 and 20% of the NSAID by day 30 and all or substantially all of the product by day 80. Some of the disclosed systems and compositions will release between 10 and 20% of the NSAID by day 60.

The compositions or systems may comprise 10-90% by weight of the biodegradable polymer. In some embodiments, the composition or system comprises 10-20% by weight, 10-30% by weight, 10-40% by weight, 10-50% by weight, 10-60% by weight, 10-70% by weight, 10-80% by weight, 20-30% by weight, 20-40% by weight, 20-50% by weight, 20-60% by weight, 20-70% by weight, 20-80% by weight, 20-90% by weight, 30-40% by weight, 30-50% by weight, 30-60% by weight, 30-70% by weight, 30-80% by weight, 30-90% by weight, 40-50% by weight, 40-60% by weight, 40-70% by weight, 40-80% by weight, 40-90% by weight, 50-60% by weight, 50-70% by weight, 50-80% by weight, 50-90% by weight, 60-70% by weight, 60-80% by weight, 60-90% by weight, 70-80% by weight, 70-90% by weight, or 80-90% by weight, of the biodegradable polymer.

In some embodiments, the biodegradable polymer comprises an aliphatic polyester. Aliphatic polyesters include, but are not limited to, poly(glycolic acid) (PGA), polylactic acid (PLA), PLGA, polycaprolactone (PCL), poly(butylene succinate) (PBS), Poly(p-dioxanone) (PPDO), and the like.

In some embodiments, the biodegradable polymer comprises poly(lactic-co-glycolic acid) (PLGA). The PLGA may comprise any mixture of lactic acid units and glycolic acid units. In some embodiments, The PLGA comprises more lactic acid units than glycolic acid units, or more than 50%, more than 60%, more than 70%, more than 80%, or more than 90% lactic acid units.

In various embodiments, the PLGA may comprise about 95% lactic acid units and about 5% glycolic acid units; about 90% lactic acid units and about 10% glycolic acid units; about 85% lactic acid units and about 15% glycolic acid units; about 80% lactic acid units and about 20% glycolic acid units; about 75% lactic acid units and about 25% glycolic acid units; about 70% lactic acid units and about 30% glycolic acid units; about 65% lactic acid units and about 35% glycolic acid units; about 60% lactic acid units and about 40% glycolic acid units; about 55% lactic acid units and about 45% glycolic acid units; about 50% lactic acid units and about 50% glycolic acid units; about 45% lactic acid units and about 55% glycolic acid units; about 40% lactic acid units and about 60% glycolic acid units; about 35% lactic acid units and about 65% glycolic acid units; about 30% lactic acid units and about 70% glycolic acid units; about 25% lactic acid units and about 75% glycolic acid units; about 20% lactic acid units and about 80% glycolic acid units; about 15% lactic acid units and about 85% glycolic acid units; about 10% lactic acid units and about 90% glycolic acid units; or about 5% lactic acid units and about 95% glycolic acid units.

The molecular weight of the biodegradable polymer can be a wide range of values. The average molecular weight of the polymer can be from about 1,000 to about 1,000,000 Da, about 1,000 to about 500,000 Da, about 1,000 to about 100,000 Da, about 1,000 to about 50,000 Da, about 1,000 to about 10,000 Da, about 1,000 to about 5,000 Da, about 10,000 to about 1,000,000 Da, about 10,000 to about 500,000 Da, about 10,000 to about 100,000 Da, about 20,000 to about 1,000,000 Da, about 20,000 to about 500,000 Da, about 20,000 to about 100,000 Da, about 50,000 to about 1,000,000 Da, about 50,000 to about 500,000 Da, or about 50,000 to about 100,000 Da. In some embodiments, the average molecular weight of the polymer is at least about 10,000 Da. In some embodiments, the average molecular weight of the polymer is at least about 20,000 Da. In some embodiments, the average molecular weight of the polymer is at least about 50,000 Da.

The present compositions and systems comprise a solvent. Solvents useful in the pharmaceutical compositions disclosed herein include, without limitation, a pharmaceutically-acceptable polar aprotic solvent, a pharmaceutically-acceptable polar protic solvent, and a pharmaceutically-acceptable non-polar solvent. A pharmaceutically-acceptable polar aprotic solvent includes, without limitation, dichloromethane (DCM), tetrahydrofuran (THF), ethyl acetate, acetone, dimethylformamide (DMF), acetonitrile (MeCN), dimethyl sulfoxide (DMSO). A pharmaceutically-acceptable polar protic solvent includes, without limitation, acetic acid, formic acid, ethanol, N-butanol, 1-butanol, 2-butanol, isobutanol, sec-butanol, tert-butanol, n-propanol, isopropanol, 1,2 propanediol, methanol, glycerol, and water. A pharmaceutically-acceptable non-polar solvent includes, without limitation, pentane, cyclopentane, hexane, cyclohexane, benzene, toluene, 1,4-Dioxane, chloroform, N-methyl-pyrrolidone (NMP), and diethyl ether.

In some embodiments, the compositions and systems comprise naproxen, PLGA, and n-methyl pyrrolidone. In select embodiments, the compositions and system comprise 10-20% naproxen, 30-40% PLGA, and 40-60% N-methyl pyrrolidone.

In some embodiments, the compositions and systems comprise meloxicam, PLGA, and n-methyl pyrrolidone. In select embodiments, the compositions and system comprise 10-20% meloxicam, 30-40% PLGA, and 40-60% N-methyl pyrrolidone.

Compositions and systems of the present disclosure can further comprise at least one of any suitable auxiliary, such as, but not limited to, diluent, binder, stabilizer, buffers, salts, lipophilic solvents, preservative, adjuvant, or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. In some embodiments, the compositions or systems may further comprise an additive, a preservative, a stabilizer, a buffer, an antioxidant, or a combination thereof.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars, and the like; and polysaccharides or sugar polymers), which can be present singly or in combination, comprising alone or in combination 1-99.99% by weight or volume.

Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the disclosure include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melicitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present disclosure are mannitol, trehalose, and raffinose.

The lipids used in the compositions of the present disclosure can be synthetic, semisynthetic or naturally-occurring lipids, including phospholipids, tocopherols, steroids, fatty acids, glycoproteins such as albumin, anionic lipids, and cationic lipids. The lipids may be anionic, cationic, or neutral. Exemplary lipid excipients include phospholipids. Without limitations, the phospholipids can be of natural origin, such as egg yolk or soybean phospholipids, or synthetic or semisynthetic origin. Phospholipids include: egg phosphatidylcholine (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidylcholine (SPC); SPG, SPS, SPI, SPE, and SPA; the hydrogenated egg and soya counterparts (e.g., HEPC, HSPC); and other phospholipids made up of ester linkages of fatty acids in the 2 and 3 positions of glycerol.

In addition to the NSAID, the compositions or system may comprise one or more additional therapeutic or pharmaceutically active agents. As used herein “active agent” refers to drugs, pharmaceutical substances, and bioactive agents. Active agents can be organic macromolecules including nucleic acids, synthetic organic compounds, polypeptides, peptides, proteins, polysaccharides and other sugars, and lipids. The active agents can fall under a variety of biological activity classes, such as vasoactive agents, neuroactive agents, hormones, anticoagulants, immunomodulating agents, cytotoxic agents, antibiotics, antiviral agents, antigens, and antibodies.

### 3. DELIVERY DEVICES

The disclosure also provides a pre-filled delivery device comprising the compositions and system described herein. Descriptions of the components of the compositions and systems set forth above in connection with the inventive compositions and systems are also applicable to pre-filled delivery device.

A pre-filled delivery device is a device which is filled with a composition or system prior to distribution to the end user that administers the composition. In some embodiments, the drug delivery device has separate chambers for the storage for the NSAID and the polymer and/or solvent. In this embodiment, the NSAID and the polymer and/or solvent are mixed just prior or concomitantly with administration.

The drug delivery device may include any device configured to allow administration or delivery of the disclosed compositions or systems. The drug delivery device may include, without limitation, an autoinjector, a pen, an eye/ear dropper, a dropper bottle, a syringe, a pump, or implant.

In some embodiments, the device is a syringe. A pre-filled syringe typically includes a containment container forming part of a syringe body, a plunger, and either an attached hypodermic needle or such features to allow a needle to be attached by the user prior to administration.

In some embodiments, the device is a syringe system. The syringe system may comprise a first compartment comprising the NSAID coupled to a second compartment comprising the biodegradable polymer and solvent. In some embodiments, the first compartment is a syringe. In some embodiments, the second compartment is a syringe. In select embodiments, the first and second compartments are syringes.

### 4. METHODS

The present disclosure further provides for treating or preventing a disease or disorder comprising administering an effective amount of the compositions or systems of the present disclosure.

A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the active agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo and in vitro studies.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

In some embodiments, the disease or disorder comprises inflammation. In some embodiments, the inflammation comprises acute or chronic inflammation. Acute inflammation is an initial protective response of the body to remove an injurious stimulus by maintaining tissue integrity and contributing to tissue repair. It is a part of the body's natural defense system against injury and disease, and in the absence of acute inflammation, wounds and infections would never heal and progressive destruction of the tissue would compromise the survival of the organism. Severe or prolonged noxious stimulation results in a chronic inflammatory response that leads to a progressive shift in the type of cells present at the site of tissue injury. Chronic inflammation may be characterized as the simultaneous destruction and healing of tissue from the inflammatory process, with the net result of provoking injury rather than mediating repair. As such, chronic inflammation is a disease.

In some embodiments, the disease or disorder is an inflammatory disease or disorder. Inflammatory diseases are characterized by activation of the immune system in a tissue or an organ to abnormal levels that may lead to abnormal function and/or disease in the tissue or organ. The inflammatory diseases and disorders that may be treated by the methods of the present disclosure include, but are not limited to, arthritis, rheumatoid arthritis, asthma, inflammatory bowel disease (Crohn's disease or ulcerative colitis), chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis, Hashimoto's thyroiditis, Addison's disease, polymyalgia rheumatica, Sjogren's syndrome, or Churg-Strauss syndrome.

In some embodiments, the disease or disorder comprises pain of any etiology, including acute and chronic pain, any pain with an inflammatory component, and any pain in which an NSAID is usually prescribed.

The administration may comprise any local administration to the site in need or may be performed before, after, or during surgery or other procedures at or adjacent to the desired site. In some embodiments, the administration comprises parenteral administration.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, intramedullary, intrasynovial, and intrasternal injection and infusion.

In some embodiments, the compositions may be administered via surgical incision into the subcutaneous tissue, muscular tissue or directly into specific organs.

Administration may be accomplished simply by direct administration using a needle, catheter, or related infusion/injection device (e.g., pre-filled syringes, microneedles, autoinjectors), at a single location and/or timepoint or at multiple locations and/or timepoints. Other parenteral formulations may be delivered subcutaneously or intradermally by microinjection or skin patch delivery methods.

### 5. Examples

### Example 1

### In Situ Forming Implants

Formulation Preparation As shown in Table 1, naproxen in situ forming implants (FN1-6) were prepared by dissolving PLGA in N-methyl pyrrolidone. 456 mg (FN1-5) or 510 mg (FN6) of PLGA/N-methyl pyrrolidone were weighed into a syringe. 100 mg of naproxen was weighed into another syringe. Before injecting into the release medium, the two syringes were coupled together and their contents were mixed thoroughly by moving the materials back and forth to obtain a homogeneous solution. The solution was then drawn into the liquid syringe. The two syringes were decoupled and attached with a needle for injection.

TABLE 1
Composition of prepared naproxen in situ forming implants

| Formu- | | | N-methyl |
|---|---|---|---|
| lation | Naproxen | PLGA | pyrrolidone |
| FN1 | 100 mg | 178 mg PLGA (L/G 50:50; 25KD) | 278 mg |
| FN2 | 100 mg | 178 mg PLGA (L/G 50:50; 16.8KD) | 278 mg |
| FN3 | 100 mg | 178 mg PLGA (L/G 75:25; 90KD) | 278 mg |
| FN4 | 100 mg | 178 mg PLGA (L/G 75:25; 14KD) | 278 mg |
| FN5 | 100 mg | 178 mg PLGA (L/G 85:15; 100KD) | 278 mg |
| FN6 | 100 mg | 228 mg PLGA (L/G 80:20; 24.2KD) | 282 mg |

As shown in Table 2, meloxicam in situ forming implants (FM1-2) were prepared by dissolving PLGA in N-methyl pyrrolidone. 510 mg of PLGA/n-methyl pyrrolidone solution were weighed into a syringe. 100 mg of meloxicam was weighed into another syringe. Before injecting into the release medium, the two syringes were coupled together and their contents were mixed thoroughly by moving the materials back and forth to obtain a homogeneous suspension. The mixed suspension was then drawn into the liquid syringe. The two syringes were decoupled and attached with a needle for injection.

TABLE 2
Composition of prepared meloxicam in situ forming implants

| Formu- | Meloxi- | | N-methyl |
|---|---|---|---|
| lation | cam | PLGA | pyrrolidone |
| FM1 | 100 mg | 228 mg PLGA (L/G 50:50; 16.8KD) | 278 mg |
| FM2 | 100 mg | 228 mg PLGA (L/G 80:20; 24.2KD) | 278 mg |

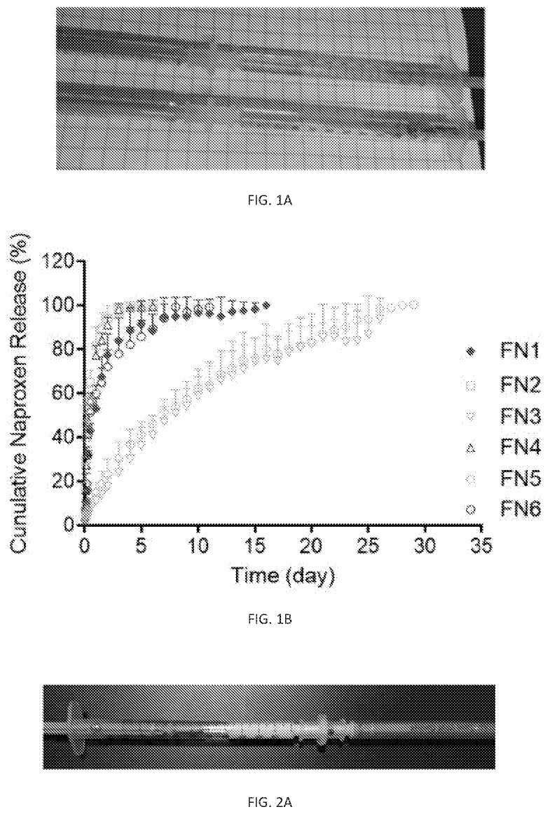

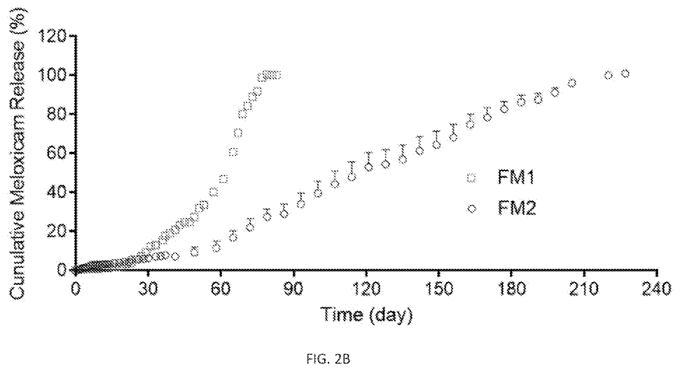

In vitro release testing of in situ forming implants The in vitro release testing of the prepared NSAID in situ forming implants was performed using USP apparatus 2 (paddle) at 37° C. with pH 7.4 phosphate buffer saline containing sodium azide as the release medium. The formulations were injected into the release medium. The in vitro release profiles are plotted in FIGS. 1B and 2B. The different naproxen in situ forming implants can release naproxen for a period of days to 1 month. The meloxicam in situ forming implants can release meloxicam for a period of 2.5 months to more than 7 months.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1-27. (canceled)

28. An injectable composition comprising:
(i) a non-steroidal anti-inflammatory drug (NSAID);
(ii) a biodegradable polymer comprises poly(lactic-co-glycolic acid) (PLGA); and
(iii) a non-polar solvent
wherein the injectable composition will form a solid biodegradable drug depot following a parenteral administration of the injectable composition to a subject in need and the solid biodegradable drug depot provides for the prolonged release of the NSAID for at least one day after administration of the injectable composition.

29. The injectable composition of claim 28, comprising 30-40% by weight of the biodegradable polymer.

30. The injectable composition of claim 28 comprising 10-20% by weight of the NSAID.

31. The injectable composition of claim 28, wherein the PLGA comprises at least 50% lactic acid units.

32. The injectable composition of claim 28, wherein the PLGA comprises at least 75% lactic acid units.

33. The injectable composition of claim 28, wherein the biodegradable polymer has an average molecular weight of at least 10 kDa.

34. The injectable composition of claim 28, wherein the biodegradable polymer has an average molecular weight of at least 20 kDa.

35. The injectable composition of claim 28, wherein the biodegradable polymer has an average molecular weight of at least 50 kDa.

36. The injectable composition of claim 28 wherein the non-polar solvent comprises pentane, cyclopentane, hexane, cyclohexane, benzene, toluene, 1,4-doixane, chloroform, N-methyl-pyrrolidone (NMP) and diethyl ether.

37. The injectable composition of claim 36 wherein the non-polar solvent comprises NMP.

38. The injectable composition of claim 28 further comprising a pharmaceutically acceptable auxiliary selected from the group consisting of a diluent, a binder, a stabilizer, a buffer, a lipophilic solvent, a preservative and antioxidant or a combination thereof.

39. The injectable composition of claim 28 further comprising a pharmaceutical excipient selected from the group consisting of a protein, a peptide, an amino acid, a lipid, a carbohydrate or a combination thereof.

40. The injectable composition claim 28, wherein the NSAID comprises naproxen.

41. The injectable composition of claim 28, wherein the NSAID comprises meloxicam.

42. A pre-filled delivery device comprising the composition of claim 28.

43. The prefilled delivery device of claim 42 wherein the prefilled delivery device is a syringe or syringe system.

44. The syringe system of claim 43, comprising a first compartment comprising the NSAID coupled to a second compartment comprising the biodegradable polymer and solvent.

45. The syringe system of claim 44, wherein the NSAID is in powder or liquid form.

46. A method of treating a disease or disorder comprising administering an effective amount of the injectable composition of claim 28 to a subject in need thereof.

47. An injectable composition consisting of:
(i) 10-20% of a non-steroidal anti-inflammatory drug (NSAID);
(ii) 30-40% of a biodegradable polymer comprises poly(lactic-co-glycolic acid) (PLGA) wherein the PLGA comprises at least 50% lactic acid units; and
(iii) N-methyl-pyrrolidone
wherein the injectable composition will form a solid biodegradable drug depot following a parenteral administration of the injectable composition to a subject in need and the solid biodegradable drug depot provides for the prolonged release of the NSAID for at least one day after administration of the injectable composition.
